(19) United States
(12) Patent Application Publication
(10) Pub. No.: US 2019/0307118 A1
(43) Pub. Date: Oct. 10, 2019

(54) SINGLE-STEP VITRIFICATION METHODS

(71) Applicant: UNIVERSITE DE LIEGE, Angleur (BE)

(72) Inventors: Delphine Connan, Liege (BE); Fabien Ectors, Liege (BE); Pierre Vanderzwalmen, Liege (BE); Luc Grobet, Liege (BE)

(73) Assignee: UNIVERSITE DE LIEGE, Angleur (BE)

(21) Appl. No.: 16/470,268

(22) PCT Filed: Dec. 15, 2017
(86) PCT No.: PCT/EP2017/083123

§ 371 Date: Jun. 17, 2019

(30) Foreign Application Priority Data
Dec. 27, 2016 (BE) BE20165981
Dec. 27, 2016 (EP) 16206907.4

(51) Int. Cl. A01N 1/02; F25B 19/00

(52) U.S. Cl. CPC A01N 1/0221; F25B 19/005

(57) ABSTRACT

A method for the cryopreservation of biological material comprising a single step of exposing the biological material to a vitrifying solution enriched with at least one cryoprotectant, for a period of less than 90 sec before cooling to a temperature for cryopreservation of the biological material.

The invention relates to a method for the cryopreservation of biological material, more particularly a single-step method for the vitrification of biological material.

### PRIOR ART

A cryopreservation method is a method for preserving biological material at very low temperature, typically 77K or −196° C., that is to say the boiling point of liquid nitrogen. A distinction is made between the slow freezing method and the vitrification method.

The vitrification method is a method of conversion, without crystallization, of a liquid into an amorphous solid. It allows the cooling of the biological material and the medium thereof to a temperature of −196° C. without the appearance of either intracellular or extracellular ice crystals. The vitrification method involves abruptly immersing the preconditioned biological material in liquid nitrogen, which, depending on the thermal inertia linked to the material itself and to its container, brings about cooling rates ranging from 900 to 20 000° C. per minute (Vanderzwalmen et al., 2010, gynecol. Obstet. Fertil., 38, 541-546). Conversely, any method of cryopreservation involving slow cooling rates of about 0.5 to 4° C. per minute is part of the slow freezing technique which involves extracellular water crystallization.

The cryopreservation method, in particular the vitrification method, generally applies to biological material of the human, animal or plant cell type, and more particularly to cells with a high individual value, such as embryonic cells, germ cells, stem cells, induced pluripotent cells, genetically modified cells, cells that are tools used for applications such as screening, diagnosis, toxicological studies, therapeutic studies, such as vaccines, or similar applications, but also to tissues, organs, embryos, gametes and precursors thereof or any other type of biological material. The use of cells with a high individual value is considerably expanding in the fields of regenerative therapy, gene therapy, medically assisted reproduction, diagnosis, pharmaceutical research and vaccine production. The cryopreservation of these cells is essential for their storage, their transportation, their screening and their expansion both in the research field and in the biobank field, intended for industrial participants or users in the therapeutic or medically assisted reproduction fields.

In order to be effective, the cryopreservation method must allow a high recovery rate, a stability of the biological characteristics regardless of the storage time in the cooling medium such as liquid nitrogen (LN2), must be chemically and (micro)biologically safe, easy to implement and automatable, and must guarantee optimal health safety. Indeed, the storage, transportation, screening and expansion efforts prior to their use are dependent thereon. The quality and safety constraints applicable to the cryopreservation of cells for therapeutic purposes are, moreover, stated in European directives (2004/03/EC; 2006/17/EC; 2006/86/EC).

The aim of all the cryopreservation methods and therefore also of the methods for the vitrification of biological material is to obtain and maintain intracellular conditions compatible with the obtaining of a vitreous amorphous state during the cooling and reheating steps.

As regards more particularly the vitrification of biological material, to date it was accepted that the key to success depended on an optimal balance between (i) the cooling and reheating rates, (ii) the cell dehydration and (iii) the penetration of cryoprotectants (CPs) into the biological material such as cells or embryos during their successive exposure to several hypertonic vitrifying solutions having an increasing concentration of cryoprotectants (CPs).

A vitrification solution consists of various types of solutes. It may comprise one or more different cryoprotectants, such as for example propylene glycol, ethylene glycol, Ficoll, dimethyl sulfoxide (DMSO), glycerol, saccharides (saccharose or diose, trehalose, glucose, fructose, sucrose, mannose, saccharose, . . . or derivatives thereof) or a mixture thereof.

A vitrification solution may also comprise solutes called upon to maintain the integrity of the biological material, such as for example phosphate buffers such as K₂PO₄ or K₂HPO₄ in the presence of KCl, NaCl or other salts, but also the saccharides mentioned above, such as glucose, saccharose, dextrose, trehalose or derivatives thereof.

Finally, a vitrification solution may also comprise solutes such as human or animal serum, such as fetal bovine serum (FBS), or fetal calf serum (FCS) or bovine serum albumin (BSA) used for their provision of protein and their cryoprotective effect.

All these solutes also play a role in the mechanism of equilibration toward an isotonic osmotic pressure.

A vitrification solution is described as hypertonic or hyperosmotic with respect to another solution of the intracellular medium which is hypotonic or hypoosmotic, separated from the vitrifying solution by a biological or semi-permeable membrane; if the solute concentration of the hypertonic vitrifying solution is such that it exerts a pressure that is lower than that exerted by the solution of the intracellular medium (hypotonic) on this membrane. This results in an attraction of water from the intracellular membrane to the vitrifying solution and/or an exchange of solutes through the membrane in order to reestablish the equilibrium of the pressures and to thus move toward isotonicity or isoosmolarity of the two solutions. A solution that is isotonic with respect to the intracellular media under physiological conditions can be described as normotonic.

The Asian Journal of Animal and Veterinary Advances 11(10) 2016 describes a two-step vitrification process in accordance with the usual practice according to the prior art. The biological material, in the case in point oocytes or embryo, is first subjected to a non-vitrifying solution containing penetrating cryoprotectants, then is exposed to a 2^{nd} vitrifying solution comprising high concentrations of penetrating and non-penetrating cryoprotectants. In the first step, the oocytes are immersed for example in a solution of 10% (v/v) ethylene glycol and 10% DMSO (v/v) in a phosphate buffer containing 18% fetal bovine serum (FBS), then in a second vitrifying solution comprising 20%% (v/v) of ethylene glycol, 20%% (v/v) of DMSO and 0.3M of trehalose in a phosphate buffer containing 18% fetal bovine serum (FBS). The two steps allow a slow equilibration between the various solutes of the intracellular and extracellular solution which equilibrate through the cell wall. This equilibration is a slow reaction of about 10 minutes (cf. page 611, last paragraph, left column)

According to the prior art, during the implementation of the vitrification method, the final solution or vitrifying solution that will be subjected to the cooling step must be a vitrifying hypertonic solution (VS), that is to say a solution containing a mixture of penetrating and/or non-penetrating CPs. The role of the VS is to coat the biological material, such as the cell, the embryo or the like, in a vitrifying sheath which inhibits the appearance of extracellular ice crystals. In practice, most of the vitrification methods comprise several steps of exposure of the biological material such as the cells or embryos, consisting of solutions of increasingly concentrated penetrating CPs before the final exposure in the VS solution. Thus, Vanderzwalmen et al., in an article in Human Reproduction from April 2013 describes, for oocytes and embryos, a first series of exposures to non-vitrifying solutions (nVSi) having an increasing concentration of penetrating CPs (of about from 3 to 4 M). The oocytes and embryos are then exposed to a vitrifying solution (VS), also known as vitrification solution (VS) comprising a high concentration of penetrating CPs (of about from 5 to 6.5M) and of non-penetrating CPs (of about 0.5 to 1M), before being immersed in the liquid nitrogen. It is known and accepted by those skilled in the art that increasingly high concentrations of cryoprotectants (CPs) during the nVSi exposure steps are necessary in order to prepare for an intracellular vitrification state. On the other hand, the mixture of penetrating and non-penetrating CPs in VS is responsible for the final cell dehydration which concentrates the intracellular components, including the salts, proteins, organelles, polysaccharides and optionally CPs that would have already penetrated into the cell during the preceding steps.

Among the non-penetrating cryoprotectants (CPs) (or those considered to be non-penetrating given the very low membrane permeability against them), are for example Ficoll®, saccharose, trehalose, lactose, mannitol, maltose, mannose and any molecule belonging to the family of disaccharides and trisaccharides or polysaccharides or polyalcohols or other derived or similar molecules.

Among the penetrating cryoprotectants, mention will for example be made of dimethyl sulfoxide (DMSO), ethylene glycol (EG), propylene glycol (PG), polyethylene glycol, triethylene glycol, glycerol and other derived or similar molecules.

While the presence of cryoprotectants (CPs) is necessary, it must nevertheless be accepted that all CPs are potentially toxic and particularly the penetrating CPs. Their toxicity depends on the concentration used, on the exposure temperature and duration, on the tonicity of the medium, on the mode of contact of the cells with the products and, finally, on the cell type. For example, dimethyl sulfoxide (DMSO), glycerol and more especially propylene glycol (PROH) can form potentially toxic formaldehyde by non-enzymatic reactions. Furthermore, DMSO is thought to have a toxic effect by destabilizing membrane proteins and displacing the bound water that is linked thereto. While everyone agrees that all CPs are toxic and that it would be beneficial to reduce their intracellular concentration, there is currently no consensus as to how to go about cryopreserving cells or embryos without using them.

### SUMMARY OF THE INVENTION

A novel method of vitrification devoid of the succession of steps of exposure to non-vitrifying solutions (nVSi) specific to the known vitrification methods has now been found, thereby making it possible to avoid the gradual exposure of cells and embryos to increasingly high concentrations of penetrating and toxic CPs.

The present invention in fact relates to a single-step method for cryopreservation, more particularly a single-step method for vitrification with exposure of biological material, for a period limited over time and before cooling, to only one vitrifying solution (VS) comprising penetrating and non-penetrating CPs. The duration of exposure is preferably less than 90 sec, and preferentially between 30 sec and 90 sec. The cooling is carried out at a temperature for cryopreservation of the biological material. The cryopreservation temperature may for example be the temperature of liquid nitrogen. The non-penetrating cryoprotectants (CPs) are present in a concentration ranging from 10% (v/v) to 60% (v/v), preferably 60% in the vitrifying solution (VS).

The penetrating cryoprotectants CPs are present in a concentration ranging from 5% to 50% (v/v) in the vitrification solution (VS), preferably 20% (v/v).

Animal or human serum, such as albumin, is also used as cryoprotectant in low concentrations ranging from 0.1% to 1% (v/v), preferably 0.6% in the vitrifying solution (VS).

Surprisingly, this single-step vitrification method makes it possible to obtain results that are equivalent to or better than those obtained with the vitrification methods according to the prior art combining successive exposures to nVSis and to VS.

This method also has the advantage of eliminating the toxic effects associated with prolonged exposures to CPs.

The biological material according to the invention may for example be any type of cells or tissues or organs, or single-cell organism or multicellular organism. In one preferred embodiment of the invention, the biological material is an embryo, embryonic cells or other related or derived cells, and also induced pluripotent cells and adult tendon mesenchymal stem cells.

The preferred biological material is the embryo or the remaining cell that is normal (for example a mesenchymal stem cell) or that has been genetically modified (for example induced pluripotent cell).

The biological material according to the invention also comprises, in terms of embryos, their zygotes, morulas or blastocysts; in terms of cells derived from embryos, the embryonic stem cells, trophoblastic cells; in terms of adult stem cells or differentiated cells from different origins, umbilical cord blood cells, cells originating from various tissues, such as blood—Peripheral Blood Monocytes —, muscle—myocytes or myoblasts, satellite cells—, ligaments and tendons—tenocytes, mesenchymal stem cells—, bones—osteoblasts, osteocytes, osteoclasts—, cartilage—chondroblasts and chondrocytes—, heart—ardiomyocytes, cardiomyoblasts—, lung and respiratory pathways—pneumocytes, ciliated cells—, liver—hepatocytes—, pancreas—alpha and beta cells, cells of the exocrine pancreas—, spleen—splenocytes, dendritic cells—, lymphoid organs, kidneys, nervous tissue—neuroblasts and neurocytes, microgliocytes, Schwann cells, interneurons—, vessels—endothelial cells, smooth muscle cells—, sense organs—corneal cells, neurosensory cells of the retina, cells of the internal ear—, stomach—gastric epithelium cells—, intestines—enterocytes, smooth muscle cells, nerve cells—, reproductive system—follicular cells, Sertoli cells, Leydig cells, primordial germ cells, stem cells and gonocytes—, cavity walls—mesothelial cells—, connective tissue—mesenchymal stem cells, fibroblasts—, thymus, thyroid, parathyroid, adrenal glands, and also the genetically modified or reprogrammed variants of these cells.

In addition to simplifying the conventional protocol, the method according to the invention makes it possible, before the cooling for the cryopreservation, to expose the biological material, such as embryos or cells, to a single vitrification solution (VS) for a short period of time, preferably less than 90 seconds and preferentially between 30 sec and 90 sec, so as to induce optimal dehydration without involving intermediate solutions (nVSi) normally used in conventional vitrification techniques. In the vitrification method according to the invention, the hypertonic solution, also called hyperosmotic solution, responsible for this rapid dehydration allows the intracellular free water to disappear and the biological material to survive following the vitrification induced by cooling. In the method according to the invention, it is no longer necessary to have recourse to cryoprotectants (CPs) which enter the intracellular space, which has the advantage of eliminating the known or unknown, short-, medium-, or long-term toxic, including genotoxic, effects linked to prolonged exposures of the intracellular medium to CPs. In the method according to the invention, the cooling rate appears to be much less critical than in the conventional method according to the prior art.

In one embodiment of the invention, the method according to the invention also comprises a step of vitrification of the biological material on a support by immersion in a cooling medium. The cooling medium may for example be liquid nitrogen.

The cryopreservation method according to the invention preferentially comprises the following steps:

a) bringing the biological material into contact with the hypertonic or hyperosmotic vitrifying solution (VS) for a period limited over time, preferably less than 90 sec;
b) deposition of the biological material resulting from step a), on a support;
c) vitrification of the biological material on the support resulting from step b) in the cooling medium which is preferably liquid nitrogen.

The biological material can thus be preserved, for example in liquid nitrogen as cooling medium, under aseptic or non-aseptic conditions for a limited period of time.

In the case of a non-aseptic vitrification, the biological material is deposited on a support, preferably in the form of a gutter, then directly immersed in the cooling medium which is preferably liquid nitrogen, after an exposure of short duration within the vitrifying solution (VS).

In the case of an aseptic vitrification, the biological material is deposited on a support after exposure to the vitrifying solution (VS) and is introduced into a container or a protective straw sealed at one end. The protective straw is sealed at its other end when it is immersed in the cooling medium which is preferably liquid nitrogen.

The protective straw must be sterile and resistant to storage at low temperature. It is preferably made of synthetic material and may consist of plastic based on polymers such as polypropylene or based on resin such as an ionomer resin. The volume of the straw may range between 250 μl and 500 μl. It is preferably 250 μl.

In one preferred embodiment of the invention, the vitrifying solution (VS) is preferably cooled to a temperature between 5 and 1° C., preferably 4° C., before being brought into contact with the biological material.

After its time spent in the cooling medium such as liquid nitrogen, the biological material is then recovered by reheating up to ambient temperature.

Contrary to the vitrification methods according to the prior art, wherein several successive baths in solutions of decreasing hypertonicities are necessary, the method according to the invention comprises only one step of abrupt reheating of the biological material having undergone the vitrification step. This abrupt reheating at the start from the cooling temperature, such as the temperature of liquid nitrogen, to ambient temperature of about 18 to 25° C. is carried out at a rate ranging from 10 000 to 30 000 degrees per minute and preferentially 20 000 degrees per minute.

The reheating is carried out by immersing the biological material in a normotonic solution such as a solution for rinsing M2 embryos (washing medium according to Quinn, J. Reprod. Fert. 1982, September:66(1):161-8).

### BRIEF DESCRIPTION OF THE FIGURE

FIG. 1 is a photo of a litter of 9 chimeric baby mice resulting from the injection of R1 mESCs vitrified in a single step according to the invention into C57BL/6 blastocysts.

### DETAILED DESCRIPTION OF THE INVENTION

The invention is illustrated below through several examples which should not be interpreted as a limitation of the invention claimed.

### Materials and Methods

1° Production of the Embryos

Five-week-old female FVB/N or C57Bl/6J inbred mice (Janvier Labs, France) were superovulated by intraperitoneal (i.p.) injection of 5 international units (i.u.) of pregnant mare serum gonadotropin (PMSG), followed 46 h later by an ip injection of 5 i.u. of human chorionic gonadotropin (hCG). The hCG injection was immediately followed by the mating of the treated females with males of identical strain. The day after the mating, the mice exhibiting a vaginal plug were euthanized by cervical dislocation and their zygotes were collected. Only the embryos having two pronuclei and a normal cytoplasm were included in this study. The in vitro development of the zygotes up to the blastocyst stage (day 5 of development in vitro) was carried out at 38° C., in 50 μl drops of culture medium for M16 embryos under oil (Whittingham, J. Reprod. Fertil Suppl. 1971, 14: 7-21), in a controlled atmosphere of 5% CO₂, saturated with moisture.

2° Use of the Embryos

In general, the embryos were randomly dispersed in various groups. The embryos developed in vitro were used at the one-cell stage (zygotes; stage 1), two-cell stage (stage II) and morula stage. After reheating, all the embryos (except for those used for the transfers) were cultured in vitro up to the blastocyst stage. For the validation of each experiment, each manipulation comprised a non-cryopreserved control group and also a group vitrified according to the “conventional” protocol, also called protocol according to the prior art described by Vanderzwalmen et al. in Human Reproduction (vol 28, 2101-2110, p 1-10, 2013), in addition to the test groups. Briefly, the vitrification according to the conventional protocol consists in exposing the embryos for 2 times 3 minutes and at ambient temperature to the non-vitrifying solutions 1 and 2 (nVS1 and nVS2), before washing them in the vitrification solution (VS) precooled to 4° C. and depositing them on their support. The latter step does not exceed 1 minute. The groups tested were directly exposed to the precooled VS before being vitrified and/or reheated according to various protocols as described below (in examples 1 to 4). The survival of the embryos is evaluated one hour after the reheating, while the development rate is evaluated on the 5th day of the in vitro culture by counting the blastocysts obtained.

3° Culture of Murine Embryonic Stem Cells (mESCs)

Murine embryonic stem cells (mESCs) of the R1 line (Nagy et al., PNAS, vol 90, p8424-8428, 1993) were cultured in gelatinized culture dishes without feeder cells, in a medium and under conditions that preserve their pluripotency and their multiplication potential. The medium used is as described in table 1 below.

TABLE 1
Composition of the mESC culture medium

| | % | ml |
|---|---|---|
| DMEM KO | 81 | 40 or 40.5* |
| SERUM | 15 | 7.5 |
| Na Pyruvate 100x | 1 | 0.5 |
| NEAA 100x | 1 | 0.5 |
| B-ME 10 mM 100x | 1 | 0.5 |
| Pen-Strep-Glut | 1 | 1.0 or 0.5* |
| 50x or 100x | | |
| mLIF (murine Leukemia | / | 0.025 |
| inhibiting factor) | | |
| TOTAL | 100 | 50.0 |

Starting from the third day after the beginning of the culture, the medium is changed daily. When the growth requires it (70% confluence), the cells are distributed at a density in accordance with the needs of the experiment in new culture dishes. To do this, the cultures are rinsed with PBS without calcium or magnesium, and then harvested using trypsin-EDTA and incubated for 3 to 5 minutes in order to obtain a homogeneous cell suspension. The trypsin activity is stopped using 6 to 8 volumes of a washing solution as described in table 2 below.

TABLE 2
composition of the mESC washing medium

| | % | ml |
|---|---|---|
| IMDM (Iscove's | 91 | 500 |
| Modified Dulbecco's | | |
| Medium) | | |
| Newborn calf serum | 10 +/− 1 | 50 |
| Na Pyruvate 100x | 1 +/− 0.1 | 5.5 |
| NEAA (Non Essential | 1 +/− 0.1 | 5.5 |
| Amino Acids) 100x | | |
| B-ME 10 mM 100x | 1 +/− 0.1 | 5.5 |
| Pen-Strep-Glut | 1 +/− 0.1 | 5.5 or 11* |
| 50 or 100x* | | |
| TOTAL | 100 | 572 |

The suspension of washed cells is centrifuged and the pellet is resuspended in culture medium. The cells are distributed into new culture dishes at a density in accordance with the needs of the experiment.

4° Cryoprotective Solutions Using the Examples According to the Invention

All the nVSi cryoprotective solutions used in the examples are prepared from a buffer solution of D-PBS (Sigma D-4031) supplemented with 10% of fetal calf serum (FCS) for cell culture originating from commercial sources (for example fetal bovine serum from) Gibco®. The nVS1 and nVS2 solutions used for the control group are prepared conventionally according to the prior art and described by Vanderzwalmen et al., in Human Reproduction (vol 28, 21.01-2110, p 1-10, 2013). The nVS1 solution contains 5% (v/v) of dimethyl sulfoxide (DMSO) and 5% (v/v) of ethylene glycol (EG), whereas the nVS2 solution contains 10% (v/v) of DMSO and 10% (v/v) of EG.

The VS hyperosmotic solution used subsequent to the nVSi solutions according to the conventional method of the prior art, or on its own in the method according to the invention, consists of 20% (v/v) of DMSO, 20% (v/v) of EG, 0.5 M of saccharose (Sigma S-1888) and 25 μM of Ficoll (Sigma F-8636). The saccharose solutions (S-1888) used are prepared from D-PBS buffer (Sigma D-4031) supplemented with 10% of fetal calf serum.

5° Cell Counting after Reheating

The mESCs are counted using a Neubauer cell, immediately, and 24 and 48 hours after they have been reheated. The mortality was estimated by means of a trypan blue excluding test.

6° Single-Step Vitrification Method According to the Invention

### Vitrification of the Embryos:

For the single-step vitrification, the embryos in groups of 4 to 6 are removed from their culture medium and are moved into a 0.5 ml drop of VS precooled to 4° C. After exposures of varying durations in the VS (30; 90; 120; 150 and 180 seconds for experiment 1; 30 versus 150 seconds for experiments 2, 3 and 4b; 50 seconds for experiments 3 and 4b), the embryos are placed on the gutter of the vitrification support: VitriPlug (Vitrimed, Austria) in the case of the non-aseptic vitrifications (examples 1, 2, 3, 4a and control group), and VitriSafe (Vitrimed, Austria) for the aseptic vitrifications (examples 3 and 4b). The term “aseptic vitrification” is intended to mean a vitrification without direct contact of the medium and the embryos with the liquid nitrogen. During the aseptic vitrifications, this VitriSafe support is introduced into a 0.3 ml protective straw (CryoBioSystem) previously identified, ballasted and sealed at one end (there are 2 compartments in the 0.3 ml straws. These 2 compartments are separated by a cottonwool plunger. The large compartment of 0.3 ml is intended to receive the VitriSafe® while the other is intended to receive a ballast (stainless steel rod coated with a colored plastic film) wrapped in an identification label). During the immersion in the liquid nitrogen, the second end of the protective straw is also sealed. During both the aseptic and non-aseptic vitrifications, the cooling is accelerated by performing circular movements in the liquid nitrogen in order to prevent the “Leidenfrost” effect which causes a reduction in the rate of cooling by formation of an insulating gas layer at the periphery of the straw.

Two control groups are treated in parallel: a group of non-cryopreserved embryos and a group vitrified according to the conventional method of the prior art described in Vanderzwalmen et al. (Human Reproduction, 2013).

### Vitrification of the Embryonic Stem Cells:

The vitrification is carried out aseptically for the vitrification according to the prior art and for the single-step vitrification according to the invention. The cells in culture are harvested as described above, and an aliquot of cell suspension is then used for a cell count, and optionally distributed into fractions as a function of their count. The cell suspension is then centrifuged, and the pellet is resuspended in 250 μl of VS solution precooled to 4° C. for 50 seconds. During this incubation, the cell suspension is suctioned into a 250 μl straw which is sealed at the two ends before being immersed in the liquid nitrogen in the same way as explained above for the embryos.

7° Reheating Step Following the Vitrification According to the Invention

During the reheating after an aseptic vitrification, and while the protective straw is still partially immersed in the liquid nitrogen, the sealed end corresponding to the compartment containing the VitriSafe® is cut with scissors and the VitriSafe is extracted therefrom. Its gutter supporting the embryos is directly and immediately immersed in a drop of 0.5 ml of 0.5 M saccharose (control group) or 0.25 M saccharose (groups of examples 1 and 4a) or in M2 culture medium at ambient temperature (examples 2 and 4b). The latter step of the reheating procedure by immersion is carried out at ambient temperature and is identical when VitriPlugs® are used. It is responsible for the reheating and also for the immediate dilution of the VS.

8° Statistical Analysis

In all the examples relating to the embryos, the statistical analysis was carried out according to a linear model (analysis of variance) using the SAS software. Only the differences for which the value of p is less than 0.05 are considered to be significant.

### EXAMPLES AND RESULTS

### Example 1: Single-Step Vitrification Step According to the Invention of Embryos at Stages I and II and the Morula Stage: Effect of the Duration of Exposure to the VS

Embryos are harvested at the one-cell stage (zygote; stage 1), according to the production method described in “Manipulating the mouse embryo, a laboratory manual” 4th edition, 2013 Behringer et al., CSH Press. Some of these embryos will be assigned to the non-cryopreserved control group and serve as a reference for the experiment. All the experiments for which this reference group gave a percentage of blastocysts of less than 90% were invalidated. Among the vitrified embryo groups, one group is treated according to the conventional method of the prior art described in Vanderzwalmen et al. (Human Reproduction, 2013) and five other groups of embryos are exposed directly to the vitrification solution (VS) described in point 3 for respectively 30 sec, 90 sec, 120 sec, 150 sec and 180 sec before the cooling.

After that, the embryos are placed in groups of five (+ or −1) on a non-aseptic support (from Vitrimed®) and directly immersed in the liquid nitrogen so as to be preserved therein for an extended period generally ranging from one day to one week.

During the reheating, all the groups, including the control group vitrified according to the conventional method described by Vanderzwalmen et al. in Human Reproduction (vol 28, 2101-2110, p 1-10, 2013), but except for the non-cryopreserved control group, are abruptly brought, at a rate of 20 000° C. per minute, to ambient temperature while immersing them in a solution of saccharose (Sigma S-1888) diluted in PBS (Sigma D4031) at the concentration of 0.25M. The groups of embryos remain in this solution for a varying period ranging from 1 to 3 minutes.

The embryos are then washed in the M2 medium (washing medium according to Quinn in J. Reprod. Fert. 1982, September: 66(1):161-8) before being placed in culture in the M16 medium (Whittingham, in J. Reprod. Fertil Suppl. 1971, 14: 7-21). Only the embryos recovered are taken into account. The experimental survival rate is calculated after one hour of culture. On the other hand, the development rate is evaluated on day 5 of the development. This is the blastocyst percentage obtained and expressed relative to the number of embryos recovered after the reheating.

The same experiment was then carried out with embryos harvested at the zygote stage and developed in vitro as far as the 2-cell stage after 24 hours of culture.

The same experiment was carried out with embryos harvested at the zygote stage and developed in vitro as far as the morula stage (+1-16 cells) after 36 hours of culture.

The results are reproduced in table 1 which presents the survival rates observed one hour after the reheating and the blastocyst percentages observed at the 5th day of development after single-step vitrification of the zygotes, stage II and morula stage.

During example 1, a total of 681 zygotes were harvested from 46 superovulated females.

TABLE 1
Survival rate 1 hour after reheating (T0) and percentages of blastocysts
observed after single-stage vitrification as a function of the developmental stage
(zygote, II and morula stages) and of the duration of exposure to the VS. The reheating
was carried out in 0.25M saccharose.

| | | Number of | | | |
|---|---|---|---|---|---|
| | | embryos | | | |
| | Number of | recovered | % survival (S) | % of blastocysts (B) | Number of |
| | embryos | (R) | at T0 (S/R) | on D5 (B/R) | replicas |
| Zygotes | | | | | |
| Control | 18 | 18 | 100.0 (18) | 77.8 (14)^{a} | 2 |
| Conventional | 41 | 39 | 87.2 (34) | 61.5 (24) | 4 |
| vitrification | | | | | |
| VS 30 sec | 29 | 28 | 89.3 (25) | 71.4 (20) | 3 |
| VS 90 sec | 29 | 27 | 96.3 (28) | 63.0 (17) | 3 |
| VS 120 sec | 31 | 30 | 86.7 (27) | 36.7 (11) | 4 |
| VS 150 sec | 22 | 20 | 80.0 (18) | 40.0 (8) | 3 |
| VS 180 sec | 22 | 22 | 86.4 (19) | 27.3 (6)^{b} | 3 |
| Stages II | | | | | |
| Control | 40 | 40 | 100.0 (40)^{c} | 95.0 (38)^{e} | 3 |
| Conventional | 30 | 28 | 92.9 (26) | 67.9 (19) | 4 |
| vitrification | | | | | |
| VS 30 sec | 51 | 39 | 79.5 (31) | 51.3 (20) | 5 |
| VS 90 sec | 31 | 31 | 77.4 (24) | 61.3 (19) | 4 |
| VS 120 sec | 27 | 27 | 96.3 (26) | 63.0 (17) | 3 |
| VS 150 sec | 26 | 26 | 96.2 (25) | 50.0 (13) | 3 |
| VS 180 sec | 25 | 25 | 44.0 (11)^{d} | 4.0 (1)^{f} | 3 |
| Morulas | | | | | |
| Control | 43 | 43 | 100.0 (43)^{g} | 95.3 (41)ⁱ | 4 |
| Conventional | 68 | 62 | 91.9 (57) | 87.1 (54) | 7 |
| vitrification | | | | | |
| VS 30 sec | 34 | 32 | 100.0 (32) | 84.4 (27) | 5 |
| VS 90 sec | 34 | 32 | 81.3 (26) | 62.5 (20) | 4 |
| VS 120 sec | 25 | 25 | 80.0 (20) | 68.0 (17) | 4 |
| VS 150 sec | 27 | 27 | 85.2 (23) | 70.4 (19) | 3 |
| VS 180 sec | 28 | 28 | 39.3 (11)^{h} | 0.0 (0)^{j} | 3 |

The values with different superscripts are significantly different: a:b = p < 0.05; c:d = p < 0.02; e:f = p < 0.05; g:h = p < 0.0005; i:j = p < 0.0001.

Upon analysis of this example 1, it was observed for the zygotes that the survival rates after 1 hour are not affected by the duration of exposure to the VS. The best rates of development into blastocysts are obtained with the lowest exposure durations, and therefore the lowest intracellular concentrations of CPs (ICCPs): the 30 and 90 second groups.

With regard to stages II, the survivals after 1 hour fall with exposures of 180 seconds. The amount of blastocysts obtained on the fifth day of culture are similar to those obtained for the zygotes, with an optimum centered around 90 seconds of exposure, and become very low with exposures of 150 and especially 180 seconds.

For the morulas, in the same way as for stage II, the survival rates after 1 hour are excellent up to 150 seconds, then fall drastically. The same is true for the percentages of blastocysts on the 5th day, for which the optimum is centered around the short exposures. Thus, surprisingly, for each stage studied, the rates of development are the best for the shortest durations of exposure to the VS, where they are similar to those obtained in the control groups after conventional vitrification according to the prior art. This demonstrates that a short abrupt single-step dehydration according to the invention induces intracellular vitrification and is not more harmful than a dehydration in steps which each time is followed by an entry of CPs followed by water. Thus, mouse embryo survival is possible after exposures to the VS as short as 30 seconds. This also means that the embryos survive the vitrification despite a very low or even zero intracellular concentration of CPs. Indeed, the shortest durations of exposure to the VS (30 seconds) bring about dehydration of the cell without allowing the entry of CPs or of water, which in this example resulted in a greater effectiveness of the vitrification (survival and development) compared with the longest exposures which allow the entry of CPs followed by water (Vanderzwalmen et al., Human Reproduction, vol 28, 2101-2110, p 1-10, 2013).

### Example 2: Single-Step Vitrification of Stages I and II and the Morula Stage: Effects of the Single-Step Dilution of the VS in Saccharose-Free Medium (M2 Medium Alone) During Reheating, Inducing an Instantaneous Cytoplasmic Rehydration

During the conventional vitrification process according to the prior art (Vanderzwalmen et al., Human Reproduction, vol 28, 2101-2110, p 1-10, 2013), the CPs enter the cell during the various exposures to the nVSi solutions which precede the exposure to the VS and the cooling. During the reheating, the cells were exposed to saccharose, an osmotically active agent used to counteract an abrupt and excessive entry of water into the dehydrated cytoplasm containing the penetrating CPs that enter the cell during the nVSi exposure steps. During the reheating process which follows a conventional vitrification according to the prior art, the absence of this saccharose-containing solution results in a bursting of the cell following an excessively abrupt and massive entry of water. This excess entry of water is solely linked to the presence of penetrating and osmotically active CPs inside the cell (Vanderzwalmen et al., Human Reproduction, vol 28, 2101-2110, p 1-10, 2013).

In the case of the vitrification according to the invention, the embryos are not exposed to the nVSi. There should not therefore be any entry of CPs into the cytoplasm, especially if the dehydration phase linked to the exposure to the VS is short. If this hypothesis is correct, in the absence of intracellular CPs, saccharose will no longer be necessary to prevent the excess entry of water during the reheating. Direct reheating in saccharose-free M2 medium was carried out, during example 2, in order to verify this hypothesis.

As for example 1, mouse embryos are vitrified at the zygote, 2-cell and morula stages. A non-cryopreserved control group serves as a reference for the experiment and control groups are vitrified and reheated by the prior art method described in Vanderzwalmen et al. (vol 28, 2101-2110, p 1-10, 2013). Two other groups of embryos (zygotes, stages 2 and morulas) are vitrified according to the invention by direct exposure to the vitrification solution (VS) for 30 sec or 150 sec.

After this, the embryos are placed in groups of five (+ or −1) on a non-aseptic support (VitriPlug, from Vitrimed®) and directly immersed in liquid nitrogen in order to be preserved therein for an extended period generally ranging from one day to one week.

During the reheating, the control group vitrified according to the prior art (Vanderzwalmen et al., in Human Reproduction, 2013) is abruptly brought, at a rate of 20 000° C. per minute, to ambient temperature while immersing it in a solution of saccharose (Sigma S-1888) diluted in PBS (Sigma D4031) at the concentration of 0.5 M. This group of embryos remains in this solution for a varying period of time ranging from 1 to 3 minutes, before being washed in M2 medium (washing medium according to Quinn, 1982) and then being placed in culture in M16 medium (Whittingham, J. Reprod. Fertil Suppl. 1971, 14: 7-21). The other groups vitrified according to the invention are also abruptly brought, at a rate of 20 000° C. per minute, to ambient temperature, but this time while immersing them directly in M2 medium (Quinn, 1982), before placing them in culture in M16 medium (Whittingham, J. Reprod. Fertil. Suppl. 1971 June: 14:7-21).

Table 2 presents, for zygotes, stages II and morulas, the survival rates observed 1 hour after the reheating and the percentages of blastocysts observed on the 5th day of development after single-step vitrification and single-step reheating in saccharose-free medium.

A total of 526 zygotes were harvested from 25 mice and used during example 2.

TABLE 2
Survival rates after 1 hour (T0) and amount of blastocysts on D5 after single-
step vitrification and reheating/immediate rehydration in saccharose-free M2 medium
(single-step reheating).

| | | Number of | | | |
|---|---|---|---|---|---|
| | | embryos | % survival | | |
| | Number of | recovered | (S) at T0 | % of blastocysts (B) | Number of |
| | embryos | (R) | (S/R) | on D5 (B/R) | replicas |
| Zygotes | | | | | |
| Control | 15 | 15 | 100.0 (15) | 93.3 (14) | 1 |
| Conventional | 41 | 39 | 87.2 (34) | 61.5 (24)^{b} | 4 |
| vitrification | | | | | |
| VS 30 sec | 52 | 48 | 91.7 (44) | 58.3 (28)^{b} | 5 |
| VS 150 sec | 62 | 61 | 75.4 (46) | 41.0 (25)^{b} | 4 |
| Stages II | | | | | |
| Control | 41 | 41 | 100.0 (41) | 100.0 (41) | 2 |
| Conventional | 30 | 28 | 92.9 (26) | 67.9 (19) | 4 |
| vitrification | | | | | |
| VS 30 sec | 32 | 31 | 96.8 (30) | 80.6 (25) | 3 |
| VS 150 sec | 45 | 44 | 72.7 (32) | 63.6 (28) | 3 |
| Morulas | | | | | |
| Control | 35 | 35 | 100.0 (35)^{a} | 100.0 (35)^{c} | 2 |
| Conventional | 68 | 62 | 91.9 (57) | 87.1 (54) | 7 |
| vitrification | | | | | |
| VS 30 sec | 53 | 53 | 100.0 (53) | 96.2 (51) | 3 |
| VS 150 sec | 52 | 52 | 61.5 (32)^{b} | 46.2 (24)^{d} | 3 |

The values with different superscripts are significantly different: a:b = p < 0.002; b:c = p < 0.0005; c:d = p < 0.0004.

Upon analysis of this example 2, regardless of the stage of the embryo during the vitrification, the survival rate at TO and also the rate of development into blastocysts after 5 days are equivalent or even greater (exposures of 30 seconds to the VS) for the method according to the invention (single-step vitrification and single-step reheating without saccharides) compared with the conventional vitrification. It thus appears that the osmotic activity of saccharose during the reheating after single-step vitrification according to the invention is not necessary, in particular for short exposures to the VS (30 sec). The abrupt immersion in a normotonic solution (M2) does not therefore bring about cell death following swelling thereof. This is not the case during the method according to the prior art, wherein direct reheating in saccharose-free medium drastically reduces the viability of the embryo, following excessive swelling thereof (Vanderzwalmen et al., Human Reproduction, vol 28, 2101-2110, p 1-10, 2013).

Our hypothesis according to which, contrary to the vitrification according to the prior art, the single-step vitrification according to the invention drastically limits or even eliminates the entry of cryoprotectants (CPs) into the cells is thus verified.

Moreover, in conjunction with example 1, this experiment shows that the process of single-step vitrification followed by single-step reheating according to the invention is at least as effective as the vitrification according to the prior art in regards to the viability and development of the cells, while at the same time ensuring a reduction in or even elimination of their content of potentially harmful cryoprotectants (CPs).

### Example 3: Single-Step Vitrification and Reheating: Effect of the Nature of the Support: Aseptic or Non-Aseptic

The previous examples (1 and 2) demonstrated that the single-step vitrification according to the invention results in intracellular concentrations of cryoprotectants (ICCPs) which are very low or even zero.

One of the dogmas of intracellular vitrification according to the prior art is inferred from the properties of the vitrification of aqueous media: the lower the concentration of CPs in the cell, the higher the cooling and the heating rates must be in order to generate and maintain the vitreous state (Yavin et al., Hum Reprod. 2009 April; 24(4):797-804).

During the single-step vitrification according to the invention, it is thus expected that a reduction in the cooling and reheating rate would not make it possible either to obtain or to retain the vitreous state and would cause the appearance of ice crystals, harmful to the cells.

The non-aseptic support used thus far in examples 1 and 2 allows direct contact of the biological sample with the liquid nitrogen. This results in cooling rates of about +1-20 000° C. per minute. Conversely, the aseptic support does not allow direct contact with the biological sample with the liquid nitrogen since the support carrying the embryos is placed in a protective straw. As a result of the presence of this straw, the cooling rate is slowed and is then only +1-1000° C. per minute.

In order to evaluate the effect of the cooling rate on the single-step vitrification according to the invention, the latter was carried out with the two types of support, aseptic and non-aseptic. A single reheating method was used: in a single step and directly in M2 medium.

As for examples 1 and 2, mouse zygotes are harvested and directly vitrified at this stage. A non-cryopreserved control group serves as a reference and control groups are vitrified by the conventional method of the prior art described by Vanderzwalmen et al. (Human Reproduction, vol 28, 2101-2110, p 1-10, 2013). Other groups of embryos are directly exposed to the VS for 50 sec or 150 sec.

After that, the embryos are placed in groups of five (+ or −1) either on a non-aseptic support (VitriPlug®, from Vitrimed®), or on an aseptic support (Vitrisafe®, from Vitrimed®) and directly immersed in liquid nitrogen so as to be preserved therein for a period of time generally ranging from one day to one week.

During the reheating, the control group vitrified according to the conventional method according to the prior art (Vanderzwalmen et al., Human Reproduction, vol 28, 2101-2110, p 1-10, 2013) is abruptly brought, at a rate of 20 000° C. per minute, at ambient temperature while immersing them in a solution of saccharose (Sigma S-1888) diluted in PBS (Sigma D4031) at the concentration of 0.5 M. This group of embryos remains in this solution for a varying period of time ranging from 1 to 3 minutes, before being washed in M2 medium (washing medium according to Quinn, J. Reprod. Fert. 1982, September:66(1):161-8) and then being placed in culture in M16 medium ((Whittingham, J. Reprod. Fertil Suppl. 1971, 14: 7-21). The other groups are also abruptly brought, at a rate of 20 000° C. per minute, to ambient temperature, but while immersing them directly in M2 medium (Quinn, 1982) before placing them in culture in M16 medium (Whittingham, 1971).

Table 3 presents the survival rates observed 1 hour after the reheating and the percentages of blastocysts observed on the 5th day of development.

A total of 405 zygotes were harvested and used during example 3. They were harvested from 17 mice. Because of its greater complexity, the vitrification under aseptic conditions cannot be carried out in less than 50 seconds. For this reason, this comparison is carried out over this period of time in the two “aseptic” and “non-aseptic” groups.

TABLE 3
Survival rates after 1 hour (T0) and amounts of blastocysts on D5 after
single-step vitrification of zygotes on aseptic and non-aseptic supports and
reheating/instantaneous rehydration in a saccharose-free M2 medium

| | | Number of | | % of | |
|---|---|---|---|---|---|
| | | embryos | % survival | blastocysts | Number |
| | Number of | recovered | (S) at T0 | (B) on | of |
| Zygotes | embryos | (R) | (S/R) | D5 (B/R) | replicas |
| Control | 15 | 15 | 100.0 (15) | 93.3 (14)^{a} | 1 |
| Conventional | 41 | 39 | 87.2 (34) | 61.5 (24) | 4 |
| vitrification | | | | | |
| VS 50 sec | 57 | 49 | 83.7 (41) | 73.5 (36)^{c} | 3 |
| aseptic | | | | | |
| VS 50 sec | 52 | 48 | 91.7 (44) | 58.3 (28) | 5 |
| NON-aseptic | | | | | |
| VS 150 sec | 121 | 110 | 72.7 (80) | 23.6 (26)^{b} | 3 |
| aseptic | | | | | |
| VS 150 sec | 62 | 61 | 75.4 (46) | 41.0 (25) | 4 |
| NON-aseptic | | | | | |

The values with different superscripts are significantly different a:b = p < 0.02; b:c = p < 0.02.

No significant difference is observed between the non-aseptic and aseptic groups. During short exposure (50 sec) to the VS, the two types of containers give very similar results comparable to the vitrification according to the prior art. For the two packaging forms, the longer exposures (150 seconds) have a tendency to decrease the blastocyst development, and more so for the aseptic support.

This example further shows that, during the vitrification according to the invention, it is not the intracellular presence of CPs that is linked to the intracellular vitrification.

Indeed, the shortest durations of exposure to the VS (50 seconds) bring about dehydration of the cell without allowing the entry of CPs or water (Vanderzwalmen et al., 2013), which, in the previous examples (1 and 2) resulted in a greater effectiveness (survival and development) compared with the longer exposures which allow the entry of CPs and of water (Vanderzwalmen et al., 2013).

It is commonly accepted during the vitrification according to the prior art, that reducing the cooling rate (for example linked to the use of aseptic supports) goes together with the need for a higher intracellular concentration of cryoprotectants (ICCPs). According to this principle, given the low or zero ICCPs induced by the vitrification according to the invention, a decrease in embryo survival should be observed with the aseptic supports (which considerably reduce the cooling rate). However, under the conditions of the invention wherein the ICCP is virtually zero (50 second exposure), our results do not show this reduction in survival with the aseptic supports.

In conclusion, during the vitrification according to the invention, and during short exposures (30 to 50 seconds) to the VS, the dehydration state reached by the cells is such that the cooling rate is less important for achieving and maintaining the vitreous state than during the vitrification according to the prior art. In the case of longer exposure to the VS (150 seconds) allowing the entry of CPs and of water into the cell after the initial dehydration process (Vanderzwalmen et al., Human Reproduction, vol 28, 2101-2110, p 1-10, 2013), the relationship between the ICCP and the cooling rate for achieving the vitreous state is reestablished, as suggested by the effectiveness which tends to decrease when the aseptic support is used.

### Example 4: Use of the Transfer of Zygotes into Pseudopregnant Recipient Mice in Order to Confirm the Ability of the Embryos to Become Baby Mice after the Single-Step Vitrification According to the Invention

### Example 4a: Non-Aseptic Vitrification: Effects of the Duration of Exposure to the VS on the % of Births after Transfers

The birth of young mice after transfer of single-step-vitrified embryos is the ultimate proof of the absence of toxicity of the method.

(i) In this context, zygotes harvested and vitrified at this stage after exposure to the VS for 30 and 150 seconds were transferred into the oviduct of pseudopregnant recipient mice according to the protocol described in “Manipulating the mouse embryo, a laboratory manual” 4th Edition, 2013 Behringer et al., CSHL Press.

Table 4 presents the birth percentages obtained. Groups of 154 and 103 zygotes were vitrified according to the protocol of single-step non-aseptic vitrification and exposures to the VS of respectively 30 and 150 seconds; after reheating in 0.25 M saccharose, 142 and 53 of them were transferred on the same day into pseudopregnant recipient mice.

TABLE 4
Percentages of young mice after transfers of zygotes vitrified according
to the protocol of single-step non-aseptic vitrification and exposures to
the VS of respectively 30 and 150 seconds; the reheating was carried
out in 0.25M saccharose.

| | | | | Average | |
|---|---|---|---|---|---|
| | | | | number of | |
| | Number of | | | young | |
| | embryos | Number of | % births | mice per | Number of |
| Zygotes | transferred | recipients | (n) | recipient | replicas |
| VS 30 sec | 142 | 5 | 21.8 (31) | 5.2 | 3 |
| NON | | | | | |
| aseptic | | | | | |
| VS 150 sec | 53 | 2 | 22.6 (12) | 6.0 | 2 |
| NON | | | | | |
| aseptic | | | | | |

No significant difference in the birth rate could be detected between the two durations of exposure to the VS, which is not surprising given the complexity of the entire process and the random efficiency of the transfers. Nevertheless, the birth percentages are entirely comparable to those routinely obtained under similar conditions during reimplantations of embryos vitrified according to the prior art (20.9%; 138 born/658 transferred; F. Ectors, data not shown). The vitrification according to the invention thus preserves the biological properties of the cells and is entirely compatible with normal development to birth.

### Example 4b: Birth Percentages after Transfers of Zygotes Having Undergone the Single-Step Aseptic Vitrification (50 Sec Exposure to the VS) and Instantaneous Rehydration in M2

Table 5 presents the birth percentages obtained. A group of 52 zygotes was vitrified according to the protocol of single-step aseptic vitrification with exposure to the VS of 50 seconds; the reheating was carried out directly in M2 (washing medium according to Quinn, J. Reprod. Fert. 1982, September:66(1):161-8). All the embryos were transferred on the same day into 2 pseudopregnant recipients.

TABLE 5
Percentages of young mice after transfers of zygotes vitrified according
to the protocol of single-step aseptic vitrification and exposures to the
VS of 50 seconds; the reheating was carried out directly in M2, inducing
instantaneous rehydration.

| | Number of | | % | Average number | Number |
|---|---|---|---|---|---|
| | embryos | Number of | births | of young mice per | of |
| Zygotes | transferred | recipients | (n) | recipient | replicas |
| VS | 52 | 2 | 34.6 (18) | 9.0 | 1 |
| 50 sec | | | | | |
| aseptic | | | | | |

The birth percentages are in this case also comparable to those obtained routinely (20.9%; F. Ectors, data not shown) during reimplantation of embryos vitrified according to the prior art.

Together, examples 4a and 4b (tables 4 and 5) confirm that the single-step vitrification according to the invention, optionally followed by direct reheating in M2 medium (washing medium according to Quinn, J. Reprod. Fert. 1982, September:66(1):161-8), under aseptic or non-aseptic conditions, does not impair the ability of the zygotes to ensure a normal gestation after transfers into a recipient.

### Example 5: Single-Step Vitrification and Reheating According to the Invention of Murine Embryonic Stem Cells (mESCs)

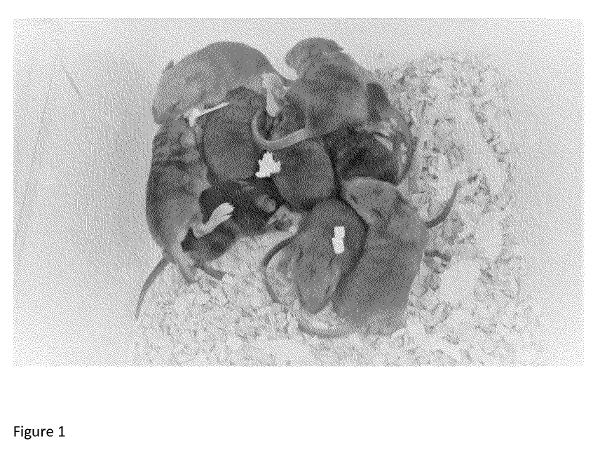

Preliminary single-step vitrification experiments according to the invention were carried out on mESCs. The conditions were not such that it was possible to perform a reliable quantification of the cell survival rates, but it appears that they are comparable to those obtained during a conventional vitrification according to the prior art. In addition, mESCs (R1 of Nagy, agouti-colored 129SV line) vitrified in a single step according to the invention were microinjected into the blastocoel of blastocysts of C57BL/6j mice and gave, on the 9 baby mice born, an extremely high percentage of chimerism (close or equal to 100%!), the coat of the chimeras showing a uniform agouti color over their entire body surface (FIG. 1).

The germline transmission of the cells derived from the vitrified line was confirmed by crossing. It thus appears that, despite the preliminary nature of this experiment, cells with extremely complex biology do not experience an impairment of said biology by the single-step vitrification according to the invention.

### DISCUSSION AND CONCLUSIONS

During preimplantation embryonic development, the more an embryo divides, the smaller its cells are, and the higher its surface/volume ratio, which enables faster transmembrane exchanges. Furthermore, the membrane permeability increases due to the appearance of new aquaporins. It should be noted that the cells present at the morula stage (+/−16 cells) have a surface/volume ratio and general biological properties (physiology) that are entirely comparable to most of the other mammalian cells. During a single-step exposure of the blastomeres to the vitrification solution (VS) according to the invention, a smaller cell reaches its maximum dehydration level more rapidly. The penetrating cryoprotectants (CPs) present in cryoprotective solutions can then enter, followed by water. Table 1 shows a decrease in the development on D5 for exposures to the VS of 150 and 180 sec, more particularly for the more advanced stages with smaller cells (morulas). If the cells survive the vitrification according to the invention after a short exposure to the VS, although there are very few (or absolutely no) CPs in their cytoplasm (see tables 1 and 3), this confirms that their survival (and therefore the quality of the vitrification) is not at all conditioned, or is conditioned very little, by the entry of CPs. Our results, showing the high efficiency of a short exposure to the VS, suggest that an absence of crystallization prevails under these vitrification conditions according to the invention. The vitrified state is very probably obtained and maintained therein both during the cooling and during the reheating, despite a low or zero ICCP. After 30 seconds of exposure to the VS, the intracellular concentration of cryoprotectants (ICCPs) can in fact only be very low or even zero. Indeed, the shortest durations of exposure to the VS (30 seconds) bring about dehydration of the cell without allowing the entry of CPs or of water, which in this example resulted in a greater efficiency of the vitrification (survival and development) compared with the longer exposures which allow the entry of CPs followed by water (Vanderzwalmen et al., Human Reproduction, vol 28, 2101-2110, p 1-10, 2013). This very low transmembrane penetration of CPs is further reduced by the precooling to 4° C. of the VS before exposure of the embryos.

During example 2 (single-step vitrification and direct reheating in M2 leading to instantaneous rehydration; table 2), we have reinforced the hypothesis of the (virtual) absence of intracellular CPs from the viewpoint of the good survival rates observed despite direct reheating in a normotonic solution (the M2 medium—washing medium according to Quinn, J. Reprod. Fert. 1982, September:66(1):161-8—without saccharose). It in fact appears that the additional osmotic activity of the saccharose during the reheating after the single-step vitrification according to the invention is not necessary, in particular for the short exposures to the VS (30 sec). The abrupt immersion of the cells in a normotonic solution (M2, washing medium according to Quinn, J. Reprod. Fert. 1982, September:66(1):161-8) does not therefore lead to cell mortality subsequent to the osmotic swelling of the cells, which does not therefore lead to an abnormal increase in their volume. This is not the case during the method according to the prior art, wherein direct reheating in saccharose-free M2 medium drastically decreases the viability of the embryo, following excessive swelling thereof (Vanderzwalmen et al., Human Reproduction, vol 28, 2101-2110, p 1-10, 2013).

Our hypothesis according to which, contrary to the vitrification according to the prior art, the single-step vitrification according to the invention drastically limits or even eliminates the entry of CPs into the cells is thus verified.

Moreover, in conjunction with experiment 1, this experiment 2 shows that the process of single-step vitrification/single-step reheating according to the invention is at least as efficient as the vitrification according to the prior art with regard to the viability and development of the cells, while at the same time ensuring a reduction in or even an elimination of their content of CPs that are potentially harmful in the short, medium or long term.

Example 3, wherein aseptic and non-aseptic supports are used, makes it possible to evaluate the effect of the cooling rate during the vitrification according to the invention. The results obtained are unexpected in that they do not correspond to what is observed during the vitrification according to the prior art, wherein the cooling rate is critical to avoid crystallization. It is in fact commonly accepted during the vitrification according to the prior art that reducing the cooling rate (for example linked to the use of aseptic supports) goes together with the need for a higher ICCP. In example 3 on the other hand, no significant difference is observed between the non-aseptic and aseptic groups, which allow very different cooling rates (+/−20 000° C. per minute for the non-aseptic support compared with +/−1000° C. per minute for the aseptic). Indeed, during short exposures (50 sec) to the VS, the two types of containers give very similar results comparable to the vitrification according to the prior art. For the two forms of packaging, the longer exposures (150 seconds) have a tendency to reduce the development into a blastocyst, but more so for the aseptic support. According to commonly accepted principles, given the low or even zero intracellular concentrations of cryoprotectants (ICCPs) induced by the vitrification according to the invention, a decrease in embryo survival should be observed with the aseptic supports (which considerably reduce the cooling rate). However, under the conditions of the invention wherein the ICCP is virtually zero (50 second exposure), our results do not show this decrease in survival with the aseptic supports. This third example reveals that, during the vitrification according to the invention, and during short exposures (30 to 50 seconds) to the VS, the dehydration state reached by the cells is such that the cooling rate is less important for reaching and maintaining the vitreous state than during the vitrification according to the prior art. In the case of longer exposure to the VS (150 seconds) allowing the entry into the cell of CPs followed by water after the initial dehydration process (Vanderzwalmen et al., Human Reproduction, vol 28, 2101-2110, p 1-10, 2013), the relationship between the ICCP and the cooling rate for achieving the vitreous state is reestablished, as suggested by the efficiency which tends to decrease when the aseptic support is used.

Our conclusions regarding the efficiency and innocuousness of a vitrification according to the invention with short exposure to the VS before cooling and direct dilution in M2 washing medium are further reinforced by the birth rates obtained after transfers (tables 4 and 5). The average percentages of baby mice routinely obtained after transfers of zygotes that are non-cryopreserved and vitrified according to the prior art are respectively 20.2% and 20.9% (F. Ectors, results not shown). Together, examples 4a and 4b show comparable efficiencies of the vitrification according to the invention which do not impair the ability of the zygotes to ensure a normal gestation after transfers into a recipient.

This demonstrates that both the instantaneous dehydration and the instantaneous rehydration preceding and following, respectively, the cooling and the reheating during a single-step vitrification according to the invention are not harmful to the various stages of development.

In the light of our results obtained in vitro but also after embryo transfers, we think we can confirm that, after exposure of the embryos to the VS for 30 sec, that is to say at the moment corresponding to the maximum cell dehydration (Vanderzwalmen et al., Human Reproduction, vol 28, 2101-2110, p 1-10, 2013), the vitrification (and therefore the viability) is not linked to the presence of CPs in the cell, but is indeed due to the absence of osmotically active water, also known as free or vicinal water.

Thus, the cell dehydration linked to the exposure to the VS for a period of time as short as 30 seconds at 4° C. does not allow CPs to enter, but is sufficient to dehydrate the cell and to bring it to conditions compatible with its survival during the various steps of the vitrification. It appears that the presence of proteins, salts, macromolecules, organelles and polysaccharides within the cell makes it possible, when said cell is sufficiently dehydrated, to form a vitreous solid state during the cooling by infinitely increasing the intracellular viscosity. There thus appear to be two types of vitreous states involved in our experiments: (i) an intracellular vitreous state linked to the absence of vicinal water associated with the transformation of the cytoplasmic gel into a vitreous solid during the cooling, and (ii) a vitreous state of the extracellular medium that has a lower concentration of macromolecules and polysaccharides and wherein the amorphous solidification of the water requires the presence of CPs at high concentrations. This demonstrates that, as long as the conditions of an extracellular vitrification are met, and the cell dehydration is sufficient, the presence of intracellular CPs is not essential to cell survival during the entire vitrification process.

The examples and validations of the vitrification according to the invention were carried out on embryos at the zygote stage and at the morula stage. It should be recalled that the cells present in the morula stage (+/−16 cells) have a surface/volume ratio and general biological properties (physiology) that are entirely comparable to most other mammalian cells, which makes it possible to extrapolate the results to these other cell types. Moreover, experiments on these cells (mESCs R1) confirm that the single-step vitrification according to the invention is efficient on other cells without impairing the biology thereof.

In conclusion, the vitrification according to the invention is based on an unprecedented approach involving a cell dehydration eliminating the free water without penetration of CPs. In addition to a methodological simplification without impairment of the efficiency compared with the conventional protocol according to the prior art, it has the advantage of eliminating the known or unknown, short-, medium- or long-term toxic, including genotoxic, effects linked to prolonged exposures of the intracellular medium to CPs.

1. A method for the cryopreservation of biological material comprising a single step of exposing the biological material to a hyperosmotic vitrifying solution enriched with at least one cryoprotectant, for a period of less than 90 sec before cooling to a temperature for cryopreservation of the biological material.

2. The method as claimed in claim 1, also comprising a step of vitrification of the biological material on a support, by immersion in a cooling medium.

3. The method as claimed in claim 1, characterized in that the biological material is an embryo.

4. The method as claimed in claim 1, characterized in that the biological material comprises embryonic cells or other related or derived cells.

5. The method as claimed in claim 1, characterized in that the vitrifying hyperosmotic solution comprises a polysaccharide derivative in the presence of saccharose.

6. The method as claimed in claim 1, characterized in that the cryoprotective agent is a mixture of dimethyl sulfoxide (DMSO) and ethylene glycol.

7. The method as claimed in claim 6, characterized in that the DMSO/ethylene glycol ratio is 50:50.

8. The method as claimed in claim 1, characterized in that the vitrifying solution also comprises animal or human serum as cryoprotectant.

9. The method as claimed in claim 1, comprising more particularly the following steps:
a) bringing the biological material into contact with a hyperosmotic vitrifying solution for a period of less than 90 sec;
b) deposition of the biological material resulting from step a) on a support; and
c) vitrification of the biological material deposited on a support in the cooling medium.

10. The method as claimed in claim 9, characterized in that the biological material deposited on the support is introduced into a straw sealed at one end, before being immersed in the cooling medium.

11. The method as claimed in claim 1, characterized in that the cooling medium is liquid nitrogen.

12. The method as claimed in claim 1, characterized in that the vitrifying solution is precooled to a temperature between 5 and 1° C., preferably 4° C., before being brought into contact with the biological material.

13. The method as claimed in claim 1, also comprising a single step of abrupt reheating, of the biological material resulting from the vitrification, to ambient temperature in a normotonic solution.

14. The method as claimed in claim 13, characterized in that the abrupt reheating is carried out at a rate of 20 000° C. per minute.
